(19) United States
(12) Patent Application Publication
(10) Pub. No.: US 2003/0186289 A1
(43) Pub. Date: Oct. 2, 2003

(54) Model cell systems for screening of anti-hypertrophic therapeutics

(75) Inventors: Erik Bush, Westminster, CO (US); Nikos Pagratis, Boulder, CO (US)

Correspondence Address: FULBRIGHT & JAWORSKI L.L.P., A REGISTERED LIMITED LIABILITY PARTNERSHIP, SUITE 2400, 600 CONGRESS AVENUE, AUSTIN, TX, 78701-3271, US

(73) Assignee: Myogen, Inc.

(21) Appl. No.: 10/348,598

(22) Filed: Jan. 21, 2003

Related U.S. Application Data
(60) Provisional application No. 60/356,070, filed on Feb. 11, 2002.
(60) Provisional application No. 60/351,076, filed on Jan. 22, 2002.

(51) Int. Cl. C12Q 1/68; G01N 33/574

(52) U.S. Cl. 435/6; 435/7.23

(57) ABSTRACT

The present invention identifies the rat muscle cell line H9C2 as a suitable platform for drug screening of potential anti-hypertrophic agents. These cells exhibit gene expression patterns that are characteristic of hypertrophic cells, thus permitting a variety of in vitro screens on candidate drugs to be conducted.

### BACKGROUND OF THE INVENTION

The present application claims priority to co-pending provisional U.S. Application Serial No. 60/356,070 filed Feb. 11, 2002 and U.S. Application Serial No. 60/351,076 filed Jan. 22, 2002. The entire text of the above referenced applications are incorporated herein by reference and without disclaimer.

1. Field of the Invention

The present invention relates generally to the fields of cardiology and molecular and cellular biology. More particularly, it concerns compositions and methods for the screening of compounds for activity against cardiac hypertrophy.

2. Description of Related Art

Hypertrophy is a growth response of muscle cells to environmental stimuli. Since muscle cells are terminally differentiated, their growth response is associated with increase in cell size and cell mass without cell division. Increase in cell mass is mainly due to elevated rates of protein synthesis. Cell hypertrophy occurs following insult or injury in the heart leading to overall cardiac hypertrophy. Cardiac hypertrophy, although initially compensating for loss of cardiac function, becomes maladaptive and leads to cardiac failure. Therefore, inhibition of cardiac myocyte hypertrophy could be a therapeutic approach in heart failure.

Hypertrophy can be induced experimentally in animal models and in in vitro cell systems. The in vitro cell system of choice for inducing hypertrophy is primary neonatal ventricular rat myocytes (NVRM). To induce hypertrophy, NVRM are treated with hypertrophic agents such as phenylephrine (PE) or endothelin (ET). To date, no cell lines have been reported that respond to the same hypertrophic agents as robustly as NVRM. Cell lines offer model systems that can be used in screening programs for the identification of potential medicines. In screening for hypertrophy inhibitors, cell lines used to date need to be treated with agents that cause hypertrophy and then compounds are evaluated for their ability to inhibit such induction of hypertrophy. The need for hypertrophy induction complicates the screens in terms of assay complexity and expense.

As mentioned above, only NVRM provide the gene expression profile suitable for such screens. However, since NVRMs are primary cells, this seriously complicates screens using such cells. The utility of these current cell systems for studying cardiac hypertrophy is limited in two principle respects: (i) these are primary cells, rather than immortal cells, and therefore die in culture and (ii) these cell lines require treatment with a inducer of the hypertrophic state. Thus, there remains a need for new model screening systems for use in identifying inhibitors of cardiac disease, ideally one that relies on endogenous signals for hypertrophic activation.

### SUMMARY OF THE INVENTION

Thus, in accordance with the present invention, there is provided a method of identifying an anti-hypertrophic agent comprising (a) providing an H9C2 cell comprising an expression cassette, the expression cassette comprising a hypertrophic promoter operably linked to a reporter coding region; (b) contacting the cell with a candidate substance; and (c) measuring the effect of the candidate substance on the activity of the hypertrophic promoter, wherein a reduction in the activity of the hypertrophic promoter, as compared to hypertrophic promoter activity in an H9C2 cell not treated with the candidate substance, indicates that the candidate substance is an anti-hypertrophic agent. The method may further comprise measuring the activity of the hypertrophic promoter in the absence of the candidate substance. The hypertrophic promoter may be selected from the group consisting of ANF, α-skeletal actin, myoglobin, MCIP, α-myosin heavy chain, β-myosin heavy chain, NFAT and MEF-2. The reporter coding region may comprise a coding region for luciferase, green fluorescent protein, β-galactosidase, chloramphenicol acetyl transferase and secreted alkaline phosphatase. The cell may be stably or transiently transformed with the expression cassette. The candidate substance may be a polynucleotide or an oligonucleotide, such as a DNA molecule or an RNA molecule. The candidate substance also may be a polypeptide or a peptide, or a small molecule.

In another embodiment, there is provided a method of identifying an anti-hypertrophic agent comprising (a) providing an H9C2 cell comprising an expression cassette, the expression cassette comprising a promoter active in the cell operably linked to a coding region for an HDAC4 or 5-GFP fusion polypeptide or an NFAT-GFP fusion polypeptide; (b) contacting the cell with a candidate substance; and (c) measuring the effect of the candidate substance on the nuclear localization of GFP fluorescence, wherein an increase in nuclear localization of GFP fluorescence, as compared to nuclear localization of GFP fluorescence in an H9C2 cell not treated with the candidate substance, indicates that the candidate substance is an anti-hypertrophic agent. The method may further comprise measuring the nuclear localization of GFP fluorescence in the absence of the candidate substance. The promoter may be CMV IE or SV40 large T. The coding region may encodes an HDAC4, 5, 7 or 9-GFP fusion polypeptide or an NFAT-GFP fusion polypeptide. The cell may be stably or transiently transformed with the expression cassette. The candidate substance may be a polynucleotide or an oligonucleotide, such as a DNA molecule or an RNA molecule. The candidate substance also may be a polypeptide or a peptide, or a small molecule.

In yet another embodiment, there is provided a method of identifying an anti-hypertrophic agent comprising (a) providing an H9C2 cell comprising:

(i) a first expression cassette, the first expression cassette comprising a GAL4 promoter operably linked to a reporter coding region;

(ii) a second expression cassette, the second expression cassette comprising a promoter active in the cell operably linked to a coding region for a MEF-2 fusion protein, wherein the MEF-2 fusion partner is VP16 or GAL4; and

(iii) a third expression cassette, the third expression cassette comprising a promoter active in the cell operably linked to a coding region for an HDAC4, 5, 7 or 9 fusion protein, wherein the HDAC4, 5, 7 or 9 fusion partner is VP16 or GAL4, but different from the fusion partner for MEF-2;

(b) contacting the cell with a candidate substance; and (c) measuring the effect of the candidate substance on the activity of the GAL4 promoter, wherein an increase in the activity of the GAL4 promoter, as compared to GAL4 promoter activity in an H9C2 cell not treated with the candidate substance, indicates that the candidate substance is an anti-hypertrophic agent. The method may further comprise measuring the GAL4 activity in the absence of the candidate substance. The promoter may be CMV IE or SV40 large T. The HDAC4, 5, 7 or 9 may be fused to GAL4 and MEF-2 may be fused to VP16, or MEF-2 may be fused to GAL4 and HDAC4, 5, 7 or 9 may be fused to VP16. The cell may be stably or transiently transformed with at least one or all of the expression cassettes. The candidate substance may be s a polynucleotide or an oligonucleotide, such as a DNA molecule or an RNA molecule. The candidate substance also may be a polypeptide or a peptide, or a small molecule. The assay may also be configured to utilize MCIP and calcineurin as the interacting molecules.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Mammalian two-hybrid assay: HDAC and MEF2 interact weakly in H9C2 cells. H9 cells were transfected with the 2-hybrid constructs and 1-hybrid controls as shown and as described in the materials and methods. The data were plotted as raw luciferase units (RLU). Symbols CN, MCIP, HDAC and MEF2 are used for the 2-hybrid constructs calcineurin in pBIND, MCIP in pACT, HDAC51-664 in pACT, and MEF2 in pBIND, respectively.

FIG. 2—Subcellular localization of HDAC-GFP in stable transfected cells. H9 cells were transfected as described in the materials and methods. Stable transfectants were selected as described in the materials and methods. Individual colonies of transfected cells is shown.

FIG. 3—H9C2 cells exhibit higher endogenous levels of hypertrophic promoter activity than other muscle lineages. Various cell lines (L6, C2C12, NLTC2, H9C2) as shown were transiently transfected with promoter-luciferase contructs as shown. Following transfection luciferase activity was measured as described in the materials and methods. The data are plotted as raw lucifearase units. Symbols ANF, NFAT, MEF2, aSkAct, and Myb are used for luciferase-promoter constructs carrying the ANF promoter, NFAT binding elements from the IL2 promoter, a synthetic promoter comprised of 3 tandem MEF2 binding sites, promoter fragment from the alpha skeletal actin gene, and promoter fragment of the myoglobin gene, respectively.

FIG. 4—Exogenous calcineurin increases MEF-2 signaling in H9C2 cells. H9 cells were transfected with 3×MEF2-luciferase promoter-reporter construct. Cells were further treated with a hypertophic agonist (phenylephrin (PE), endothelin (ET) or calcineurin (CNAa)-via transfection). Luciferase units were measured and expressed as a fraction of untreated control.

FIG. 5—H9C2 promoter activity is unresponsive to the adrenergic agonists isoproterenol, norepinephrine and forskolin. H9 cells were transfected with promoter-luciferase contructs as shown and as described in materials and methods. Transfected cells were treated with hypertrophic agonists as shown. Luciferase units were measured and plotted as fraction of untreated control vs concentration of agonist used. Symbols ANF, NFAT, MEF2, aSkAct, and Myb are used as in FIG. 3.

FIG. 6—Repression of endogenous MEF-2 and α-skeletal actin promoter activity by cyclosporine, FK506, MCIP1 and HDAC5. H9 cells were transfected with promoter-luciferase constructs as shown. Transfected cells were also treated with hypertrophy inhibitors cyclosporin A (CsA), tarcolimus (FK506) and via transfection with expression constructs for MCIP1 and HDAC5. Luciferase units were measured and expressed as fraction of untreated controls. Symbols ANF, NFAT, MEF2, aSkAct, and Myb are used as in FIG. 3.

FIG. 7—Repression of endogenous MEF-2 and α-skeletal actin promoter activity by MCIP1, HDAC5 and HDAC5dm. H9 cells were infected with promoter-luciferase adenoviral constructs constructs as shown. Infected cells were also treated with hypertrophy inhibitors via transfection with expression adenoviral constructs for MCIP1 exon 4 (MCIPex4 ADV), wt HDAC5 (HDAC5wt ADV) and double mutant HDAC5 S295,498A (HDACdm ADV) or with control beta galactosidase-expressing adenovirus (β-gal ADV). Luciferase units were measured and expressed as fraction of b-gal AND-treated control. Symbols A-SK-actin-luc ADV and 3×MEF2-luc ADV) are used for recombinant adenoviruses encoding luciferase downstream of the alpha skeletal actin of 3×MEF2 element, respectively.

FIG. 8—Comparison of two-hybrid signals with HDAC5-VP16 and HDAC5dm-VP16. H9 cells were transfected with the HDAC/MEF2 2hybrid system. Two versions of the transactivation domain chimera was used, one carrying wt HDAC sequence and a second carrying double mutant S295,498A HDAC5 mutation.

FIG. 9—Typical assay plate with HDAC5-VP16 and HDAC5dm-VP16. Well A1 is in the top left. H9 cells were transfected with the 2-hybrid HDAC/MEF components in batch as described in the materials and methods and were plated in A1-H10 as well as in G11, G12, H11 and H12. H9 cells transfected with the 1 hybrid control (omitting HDAC5) were plated on A11, B11, C11, D11, E11, F11, E12, and F12. H9 cells transfected with the double mutant 2-hybrid components were plated A12-D12. Signal to noise (S/N) and assay headroom (Sds) is shown.

FIG. 10—CsA specifically dissociates the CN/MCIP interaction in H9C2 cells but not COS cells. H9 and COS cells were transfected with the CN/MCIP 2-hybrid components and treated with a dilution series of cyclosporin A (CsA) as shown. Luciferase units were measured and expressed as fraction of untreated control and compared to measurements from a one hybrid control applying the Gal4-DNA binding-VP16 transactivation domain chimera.

FIG. 11—Activity profile of compound 6 (My9766). Luciferase levels in the 1°, 2°, and NRVM is shown. The compound position is marked by vertical arrows. Dose response curves with ANF-, S6-, AMHC-cytoblot, AK release is as shown. Inhibitors of the hypertrophic response are expected to decrease levels of ANF and S6 and increase levels of AMCH. Toxic doses of the compounds are expected to cause increase on AK levels. AK was measured using a commercial kit according to manufacturers specifications.

FIG. 12—Activity profile of compound 7 (My12261). Luciferase levels in the 1°, 2°, and NRVM is shown. The compound position is marked by vertical arrows. Dose response curves with ANF-, S6-, AMHC-cytoblot, AK release, and cell area is as shown. Inhibitors of the hypertrophic response are expected to decrease levels of ANF, S6 and cell area while increase levels of AMCH. Toxic doses of the compounds are expected to cause increase on AK levels. AK was measured using a commercial kit according to manufacturers specifications.

FIG. 13—Activity profile of compound 10 (My14783). Luciferase levels in the 1°, 2°, and NRVM is shown. The compound position is marked by vertical arrows. Dose response curves with ANF-, S6-cytoblot, and AK release. Inhibitors of the hypertrophic response are expected to decrease levels of ANF and S6. Toxic doses of the compounds are expected to cause increase on AK levels. AK was measured using a commercial kit according to manufacturers specifications.

FIG. 14—Activity profile of compound 11 (My15542). Luciferase levels in the 1°, 2°, and NRVM is shown. The compound position is marked by vertical arrows. Dose response curves with ANF-, S6-cytoblot, and AK release. Inhibitors of the hypertrophic response are expected to decrease levels of ANF and S6. Toxic doses of the compounds are expected to cause increase on AK levels. AK was measured using a commercial kit according to manufacturers specifications.

FIG. 15—Activity profile of compound 12 (My16139). Luciferase levels in the 1°, 2°, and NRVM is shown. The compound position is marked by vertical arrows. Dose response curves with ANF-, S6-cytoblot, and AK release. Inhibitors of the hypertrophic response are expected to decrease levels of ANF and S6. Toxic doses of the compounds are expected to cause increase on AK levels. AK was measured using a commercial kit according to manufacturers specifications.

### DESCRIPTION OF ILLUSTRATIVE EMBODIMENTS

The molecular events that lead to hypertrophy are subject to intense study and involve integration of several signaling pathways that trigger specific gene expression programs. In heart, the hypertrophy response is linked to two major Ca⁺⁺ signaling pathways, namely the calcineurin (CN) pathway and the calcium-calmodulin activated kinases (CaMK) pathway. Activation of these two pathways leads to elevated transcription of specific genes that are active during fetal development, but are not active in the adult (the “fetal gene program”). These genes include ANF, alpha-skeletal actin, myoglobin, and beta-myosin heavy chain among others. This is presumably due, at least in part, to the activation of two transcription factors, namely NFAT and MEF2. Therefore, promoter reporter constructs containing either native promoter fragments from the genes mentioned above, or synthetic binding sites for MEF2 and/or NFAT, show high activity when introduced in NVRM under conditions that cause hypertrophy. Unfortunately, NVRM are primary cells, which makes them less than ideal candidates for a drug screening platform.

The present inventors have demonstrated that H9C2 cells, a clonal cell line derived from embryonic BDIX rat hearts, are already in a state of enhanced hypertrophic response as determined by the activity of hypertrophic promoter-reporter constructs and subcellular localization of HDAC-GFP. The H9C2 cells exhibit key molecular indications of cardiac hypertrophy—elevated levels of hypertrophic promoter activity and cytoplasmic localization of HDAC. Furthermore, this endogenous activity can be modulated by known activators and inhibitors of hypertrophy. Since the hypertrophic signaling pathways of H9C2 cells appear to be constitutively active (to some degree) in the absence of classical agonists, and since these are immortalized cardiac cells, these H9C2 cells have great utility in assays designed to discover novel inhibitors of cardiac hypertrophy. The details of the invention are described below.

I. H9C2 Cells

H9C2 is a clonal cell line derived from embryonic BDIX rat hearts by selective serial passage (Kimes and Brandt, 1976). The primary culture that gave rise to H9C2 cells was obtained from the lower half embryonic heart of a 13-day rat embryo. Thus the original culture included mostly ventricular cells. The clonally derived H9C2 cells are spindle-shaped and grow in orderly parallel arrays on the surface of culture dishes and they rarely form multinucleated cells (Kimes and Brandt, 1976). Morphologically, H9C2 cells appear like myocytes with filament organization. Originally, H9C2 cells were thought more like skeletal muscle cells, but further analysis of their properties indicated that they are more like cardiac muscle (Hescheler et al., 1991). Recently, H9C2 cells were shown to respond to vasopressin by increase in cell size and rate of protein synthesis (Brostrom et al., 2000).

H9C2(2-1) is available from the American Type Culture Collection, Manassas, Va., under catalog #CRL-1446.

II. Expression Constructs

Expression constructs are nucleic acids that contain regulatory elements facilitating the expression of a given gene product, and a nucleic acid segment encoding that gene product. Often, such constructs are included in “vectors,” carrier nucleic acid molecules into which an expression cassette can be inserted for introduction into a cell where it can be replicated. A nucleic acid sequence can be “exogenous,” which means that it is foreign to the cell or vector into which it is being introduced, or the sequence is homologous to a sequence in the cell or vector, but in a position within the host cell genome or vector backbone in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

A. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cells, promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference).

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous to the vector or host cell.

The promoter may be a strong, constitutive promoter. The cytomegalovirus immediate early (CMV IE) promoter is one such example. Other constitutive promoters that are useful in the present invention include SV40. Another class of promoters are the tissue-specific promoters or elements. These promoters show preferential or selective expression in certain cell types. Other promoters suitable for use with the present invention include α-myosin heavy chain (heart), α-fetoprotein (liver), albumin (liver), thyroglobulin (thyroid), enolase (brain), CC10 (lung), keratin (epidermis) and β-lactoglobulin (mammary gland).

Yet another type of promoter that may be used is an inducible promoter. Inducible promoters are activated by an exogenous signal that can be provided at the discretion of the user. An example of an inducible promoter is the Tet-On®/Tet-Off® Systems (Clontech). Both Tet-On® and Tet-Off® use a chimeric transactivator to activate transcription of the gene of interest from a silent promoter. The transactivator, either tTA or rtTA, is expressed in a host cell from a constitutive or tissue specific promoter. In the Tet-Off® system, tTA binds to the Tet Response Element (TRE) in the silent promoter and activates transcription in the absence of the inducer, doxycycline. In the Tet-On® System, rtTA binds to the TRE and activates transcription in the presence of doxycycline.

In the present invention, hypertrophic promoters are those promoters believed to be particularly active cells exhibiting hypertrophic gene expression patterns, and thus useful in the screening assays described herein. These promoters include ANF, α-skeletal actin, myoglobin, α-myosin heavy chain, β-myosin heavy chain, NFAT, MEF-2, SERCA and any other gene known to be up- or down-regulated in the hypertrophic heart.

B. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

C. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

D. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

E. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

F. Polyadenylation Signals In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

G. Origins of Replication

In order to propagate a vector in a host cell, the vector may contain one or more origins of replication sites (often termed “ori”), which is a specific nucleic acid sequence at which replication is initiated. Alternatively, an autonomously replicating sequence (ARS) can be employed if the host cell is yeast.

III. Methods of Gene Transfer

A. Non-Viral Delivery

As part of the present invention, it will be necessary to transfer various genetic constructs into cells, both for transient expression and stable transformation. Suitable methods for nucleic acid delivery for use with the current invention are believed to include virtually any method by which a nucleic acid (e.g., DNA) can be introduced into a cell, as described herein, or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome-mediated transfection (Nicolau and Sene, 1982; Fraley et al., 1979; Nicolau et al., 1987; Wong et al., 1980; Kaneda et al., 1989; Kato et al, 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods.

1. Microinjection

A nucleic acid may be delivered to a cell via one or more microinjections. Direct microinjection has been used to introduce nucleic acid constructs into Xenopus oocytes (Harland and Weintraub, 1985).

2. Electroporation

In certain embodiments of the present invention, a nucleic acid is introduced into a cell via electroporation. Electroporation involves the exposure of a suspension of cells and DNA to a high-voltage electric discharge. In some variants of this method, methods are employed to render the target recipient cells more susceptible to transformation by electroporation than untreated cells.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

3. Calcium Phosphate

In other embodiments of the present invention, a nucleic acid is introduced into the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

4. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

5. Sonication Loading

Additional embodiments of the present invention include the introduction of a nucleic acid by direct sonic loading. LTK⁻ fibroblasts have been transfected with the thymidine kinase gene by sonication loading (Fechheimer et al., 1987).

6. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a nucleic acid complexed with Lipofectamine (Gibco BRL), Superfect (Qiagen) or FuGene (Roche).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

7. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

8. Microprojectile Bombardment

Microprojectile bombardment techniques can be used to introduce a nucleic acid into a cell (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

An illustrative embodiment of a method for delivering DNA into a cell by acceleration is the Biolistics Particle Delivery System, which can be used to propel particles coated with DNA or cells through a screen, such as a stainless steel or Nytex screen, onto a filter surface covered with cells. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

B. Viral Methods

The ability of certain viruses to infect cells and express transgenes transiently or stably have made them attractive candidates for the transfer of foreign nucleic acids into cells. Thus, the present invention may take advantage of viral vectors to deliver reporter gene constructs or other genes relevant to cardiac drug screening methods. Non-limiting examples of virus vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the present invention as it has a high frequency of integration and it can infect non-dividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of recombinant AAV vectors are described in U.S. Pat. Nos. 5,139,941 and 4,797,368, each incorporated herein by reference.

3. Retroviral Vectors

Retroviruses are particularly useful in stable transformation of target cells due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid segment is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression generally require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al, 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

IV. Screening for Modulators

The present invention comprises methods for identifying modulators of cardiac hypertrophy. These assays may comprise random screening of large libraries of candidate substances; alternatively, the assays may be used to focus on particular classes of compounds selected with an eye towards structural attributes that are believed to make them more likely to modulate the function of relevant molecules such as calcineurin, MCIPs, HDACs, HDAC kinase, CaM kinase, GATA4, protein 1433, MEF-2 or NFAT.

At least four different assays are envisioned. First, one may measure the activity of various promoters in H9C2 cells when treated with candidate substances. This can be facilitated by the linking of various promoters to screenable markers (discussed below). Second, one may examine subcellular localization of proteins such as HDAC4/5 and MEF-2, and MCIP and calcineurin, when treated with candidate substances. This may be accomplished by using labeled antibodies that bind to the proteins or, more easily, by generating fusion proteins between HDAC4/5, MEF-2, calcineurin or MCIP and a screenable marker protein such as GFP. Third, one may use the interaction between two molecules (e.g., HDAC4/5 and MEF-2, or calcineurin and MCIP) as an indicator of the effects of candidate substances. And fourth, one may measure changes in endogenous mRNA levels of hypertrophic response genes by quantitative PCR®, hybridization or microarrays.

It will, of course, be understood that all the screening methods of the present invention are useful in themselves notwithstanding the fact that effective candidates may not be found. The invention provides methods for screening for such candidates, not solely methods of finding them.

A. Candidate Substances

As used herein the term “candidate substance” refers to any molecule that may potentially inhibit cardiac hypertrophy. The candidate substance may be a protein or fragment thereof, a small molecule, or even a nucleic acid molecule. It may prove to be the case that the most useful pharmacological compounds will be compounds that are structurally related to molecules such as calcineurin, MCIP, MEF-2, NFAT and HDAC4/5. Using lead compounds to help develop improved compounds is known as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable compounds include antisense molecules, ribozymes, aptamers and antibodies (including single chain antibodies), each of which would be specific for a target molecule. For example, an antisense molecule that bound to a translational or transcriptional start site, or splice junctions, would be ideal candidate inhibitors.

B. Assay Formats

In a first assay format, one will examine the effects of a candidate substance on promoter expression. Typically, this will involve the use of a transgene comprising a coding region for a reporter linked to the promoter of interest. The reporters may provide visual readouts (colored enzyme products, fluorescent signals) that can be monitored (optimally in a high-throughput fashion) by automated optical systems. Typical assays involve multi-well microtitres plates.

Cells containing appropriate transgene constructs are treated with a candidate substance such that the substance will cross the cell membrane. Depending on the type of candidate substance, this may involve use of various delivery vehicles including viruses, liposomes, or other vehicle. Cells are then incubated for an appropriate time period, followed by measurement of signal (either in situ or following cell lysis). Appropriate controls include H9C2 cells not treated with the candidate substance, and cells not capable of hypertrophic signaling treated with the candidate substance.

A second assay format looks at nuclear localization. Transgene constructs comprising HDAC4/5 or NFAT, fused to a screenable marker, typically a fluorescent molecule such as GFP or luciferase, are transferred into the host cell. Following treatment with the candidate substance and appropriate incubation, cells are either visualized to detect signal, or nuclei are separated from other cellular material and measured following separation. Appropriate controls include H9C2 cells not treated with the candidate substance, and cells not capable of hypertrophic signaling treated with the candidate substance.

In a third assay format, the two-hybrid system (described below) is utilized. In this assay, the effect of a candidate substance on protein-protein interactions is measured. The read-out is transcription and, thus, suitable screenable marker genes are employed such as enzymes that produce substrates or generate optically measured signals.

C. Reporter Genes

As part of the present invention, it will be useful to employ various screenable markers genes. Such marker or “reporter” genes permit assaying of gene expression levels by looking for a signal. Suitable reporter genes include enzymes that produce a screenable product. One such enzyme is β-galactosidase, when used in the presence of specific substrates. This enzyme produces a blue product that is directly proportional to the amount of enzyme present in the sample. The product can be measured optically. Another enzyme useful in screening activity is chloramphenicol acetyl transferase, or CAT.

In another embodiment, the screenable marker may be a fluorescent chemilluminescent molecule, such as firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. The gene which encodes green fluorescent protein (GFP) is contemplated as a particularly useful reporter gene (Sheen et al., 1995; Haseloff et al., 1997; Reichel et al., 1996; Tian et al., 1997; WO 97/41228). Expression of green fluorescent protein may be visualized as fluorescence following illumination by particular wavelengths of light.

Other screenable molecules include β-glucuronidase, enhanced GFP, blue fluorescent protein, secreted alkaline phosphatase, Renilla luciferase and xylene oxidase.

D. Two-Hybrid System

The yeast two-hybrid system is often used to study protein-protein interactions as points of pharmaceutical intervention. This is done by testing compounds for their ability to modulate protein interactions. The yeast two-hybrid system is outlined in U.S. Pat. No. 5,283,173 (incorporated herein by reference), and is a technique well known to those of skill in the art. Briefly, the method is designed to assess an interaction between a first test protein and a second test protein, in vivo, using reconstitution of the activity of a transcriptional activator. Two chimeric genes that express hybrid proteins are prepared. The first hybrid protein contains the DNA-binding domain of a transcriptional activator fused to the first test protein, while the second hybrid protein contains a transcriptional activation domain fused to the second test protein. If the two test proteins interact, the two domains of the transcriptional activator are brought into close proximity, resulting in the transcription of a marker gene that contains a binding site for the DNA-binding domain. An assay can be performed to detect activity of the marker gene.

All yeast two-hybrid systems share a set of common elements: (1) a construct that directs the synthesis of a “bait”; the bait is a protein which is fused to a DNA binding domain; (2) one or more reporter genes (“reporters”) with upstream DNA binding sites for the bait; and (3) a construct that directs the synthesis of proteins fused to activation domains (“prey”). Current systems direct the synthesis of proteins that carry the activation domain at the amino-terminus of the fusion, facilitating the expression of open reading frames encoded by cDNAs. DNA binding domains used in the yeast two-hybrid systems include the native E. coli LexA repressor protein (Gyuris et al., 1993) and the yeast GAL4 protein (Chien et al., 1991).

Although most two-hybrid systems use yeast, mammalian variants suitable for use in the present invention have been developed. In one system, interaction of activation tagged VP16 derivatives with a GAL4-derived bait drives expression of reporters that direct the synthesis of Hygromycin B phosphotransferase, Chloramphenicol acetyltransferase, or CD4 cell surface antigen (Fearon et al., 1992). In another system, interaction of VP16-tagged derivatives with GAL4-derived baits drives the synthesis of SV40 T antigen, which in turn promotes the replication of the prey plasmid, because the plasmid carries a SV40 origin (Vasavada et al., 1991). A wide variety of reporting constructs may be utilized.

V. Genes of Interest

Various genes of interest may be utilized in the assays, expression constructs and recombinant cells of the present invention. These are described below.

A. HDACs

Nucleosomes, the primary scaffold of chromatin folding, are dynamic macromolecular structures, influencing chromatin solution conformations (Workman and Kingston, 1998). The nucleosome core is made up of histone proteins, H2A, HB, H3 and H4. Histone acetylation causes nucleosomes and nucleosomal arrangements to behave with altered biophysical properties. The balance between activities of histone acetyl transferases (HAT) and deacetylases (HDAC) determines the level of histone acetylation. Acetylated histones cause relaxation of chromatin and activation of gene transcription, whereas deacetylated chromatin generally is transcriptionally inactive.

Six different HDACs have been cloned from vertebrate organisms. The first three human HDACs identified were HDAC 1, HDAC 2 and HDAC 3 (termed class I human HDACs). Recently class II human HDACs, HDAC 4, HDAC 5, HDAC 6, HDAC 7 and HDAC 9 have been cloned and identified (Grozinger et al., 1999). All share homology in the catalytic region. HDACs 4, 5, 7 and 9 however, have a unique amino-terminal extension not found in other HDACs. This amino-terminal region contains the MEF2-binding domain. HDACs 4, 5, 7 and 9 are involved in the regulation of cardiac gene expression and in particular embodiments, repressing MEF2 transcriptional activity. The exact mechanism in which HDAC 4, HDAC 5, HDAC 7 and HDAC 9 repress MEF2 activity is not completely understood. One possibility is that MEF2 recruits HDAC to transcriptionally active, acetylated chromatic. HDAC deacetylates histone, converting local chromatin structure to the inactive, deacetylated state, which is less accessible to the core components of the transcription complex.

B. MEF-2

A family of transcription factors, the monocyte enhancer factor-2 family (MEF2), are known to play an important role in morphogenesis and myogenesis of skeletal, cardiac, and smooth muscle cells (Olson et al., 1995). MEF2 factors are expressed in all developing muscle cell types, binding a conserved DNA sequence in the control regions of the majority of muscle-specific genes. Of the four mammalian MEF2 genes, three (MEF2A, MEF2B and MEF2C) can be alternatively spliced, which have significant functional differences (Brand, 1997; Olson et al., 1995). These transcription factors share homology in an N-terminal MADS-box and an adjacent motif known as the MEF2 domain. Together, these regions of MEF2 mediate DNA binding, homo- and heterodimerization, and interaction with various cofactors, such as the myogenic bHLH proteins in skeletal muscle. Additionally, biochemical and genetic studies in vertebrate and invertebrate organisms have demonstrated that MEF2 factors regulate myogenesis through combinatorial interactions with other transcription factors.

C. NFAT

NFATs are part of a multigene family containing four members, NFATc, NFATp, NFAT3 and NFAT4 (McCaffery et al., 1993; Northrup et al., 1994; Hoey et al., 1995; Masuda et al., 1995; Park et al., 1996; Ho et al., 1995). These factors bind the consensus DNA sequence GGAAAAT as monomers or dimers through a Rel homology domain (RHD) (Rooney et al., 1994; Hoey et al., 1995). Three of the NFAT genes are restricted in their expression to T-cells and skeletal muscle, whereas NFAT3 is expressed in a variety of tissues including the heart (Hoey et al., 1995). For additional disclosure regarding NFAT proteins the skilled artisan is referred to U.S. Pat. No. 5,708,158, specifically incorporated herein by reference. NFAT3 is a 902-amino acid protein with a regulatory domain at its amino-terminus that mediates nuclear translocation and the Rel-homology domain near its carboxyl-terminus that mediates DNA binding.

There are three different steps involved in the activation of NFAT proteins, namely, dephosphorylation, nuclear localization and an increase in affinity for DNA. In resting cells, NFAT proteins are phosphorylated and reside in the cytoplasm. These cytoplasmic NFAT proteins show little or no DNA affinity. Stimuli that elicit calcium mobilization result in the rapid dephosphorylation of the NFAT proteins and their translocation to the nucleus. The dephosphorylated NFAT proteins show an increased affinity for DNA. Each step of the activation pathway may be blocked by CsA or FK506. This implies, and the inventors' studies have shown, that calcineurin is the protein responsible for NFAT activation.

D. Calcineurin

Calcineurin is a calcium/calmodulin-regulated protein phosphatase that modulates gene expression in cardiac and skeletal muscles during development, as well as in remodeling responses such as cardiac hypertrophy that are evoked by environmental stresses or disease. It dephosphorylates the transcription factor NF-AT3, enabling NF-AT3 to translocate to the nucleus. NF-AT3 then interacts with the cardiac zinc finger transcription factor GATA4, resulting in synergistic activation of cardiac transcription. Transgenic mice that express activated forms of calcineurin or NF-AT3 in the heart develop cardiac hypertrophy and heart failure that mimic human heart disease. Pharmacologic inhibition of calcineurin activity blocks hypertrophy both in vivo and in vitro. These results define a novel hypertrophic signaling pathway and suggest pharmacologic approaches to prevent cardiac hypertrophy and heart failure.

E. MCIPs

Signaling events controlled by calcineurin promote cardiac hypertrophy, but the degree to which such pathways are required to transduce the effects of various hypertrophic stimuli remains uncertain. One molecule of interest is MCIP (myocyte-enriched calcineurin-interacting protein), which interact with calcineurin to inhibit its enzymatic activity. Expression of MCIP1 is regulated by calcineurin activity in hearts of mice with cardiac hypertrophy, as well as in cultured skeletal myotubes. In contrast, expression of MCIP2 in the heart is not altered by activated calcineurin, but responds to thyroid hormone, which has no effect on MCIP1. Because MCIP proteins can inhibit calcineurin, these results suggest that MCIP 1 participates in a negative feedback circuit to diminish potentially deleterious effects of unrestrained calcineurin activity in cardiac and skeletal myocytes. Inhibitory effects of MCIP2 on calcineurin activity may be pertinent to gene switching events driven by thyroid hormone in striated muscles.

Transgenic mice that overexpress hMCIP1 under control of the cardiac-specific, alpha-myosin heavy chain promoter showed a 5-10% decline in cardiac mass relative to wild-type littermates, but otherwise produced no apparent structural or functional abnormalities. However, cardiac-specific expression of hMCIP1 inhibited cardiac hypertrophy, reinduction of fetal gene expression, and progression to dilated cardiomyopathy that otherwise result from expression of a constitutively active form of calcineurin. Expression of the hMCIP1 transgene also inhibited hypertrophic responses to beta-adrenergic receptor stimulation or exercise training. These results demonstrate that levels of hMCIP1 producing no apparent deleterious effects in cells of the normal heart are sufficient to inhibit several forms of cardiac hypertrophy, and suggest an important role for calcineurin signaling in diverse forms of cardiac hypertrophy.

### VI. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Plasmid constructs used. Gal4 DNA-binding domain constructs were generated by cloning cDNAs encoding constitutively active murine calcineurin (residues 1-398) and full length human MEF-2 into the pBIND expression vector (Promega, Inc.). VP16 fusion constructs were created by cloning full length human MCIP1 and HDAC5 cDNAs into the pACT expression vector (Promega, Inc.). In assays, Gal4 and VP16 fusion constructs were cotransfected with pG5-luc reporter (Promega), a firefly luciferase gene under control of a multimerized Gal4 promoter sequence. Promoter reporter constructs were generated by cloning the promoter regions of ANF, α-skeletal actin, IL-2 (with multiple NFAT binding sites) and myoglobin genes upstream of a promoter-less firefly luciferase open reading frame (pGL3 plasmid, Promega, Inc.). Similarly, three consensus MEF-2 binding sites or a single TATA element were cloned into pGL3. HDAC5-GFP construct was a gift from E. Olson, UT Southwestern Medical Center, Dallas Tex.

Cells culture and transient transfections. All muscle cell lines were obtained from the American Type Culture Collection (ATCC). Cells were cultured under conditions recommended by ATCC. Transient transfections were carried out in 96-well plates (5000 cells/well) using the Fugene transfection reagent (Roche), as per manufacturer's instructions. For CsA, FK506 and agonist experiments, drugs were added at the time of transfection. Forty-eight hours post-transfection, cells were processed for luciferase measurements.

Adenoviral constructs. Recombinant adenovirus α-skeletal actin-luc, and 3×MEF2-luc were constructed using the Adeasy vector system (Qbiogene) according to manufacturers instructions. Recombinant adenovirus HDAC5 and HDAC5-S259/498A were provided by E. Olson. Recombinant adenovirus MCIPexon4 was provided by S. Williams. Recombinant adenovirus β-galactosidase was from Qbiogene. Cells were plated in 96-well plates, and 24 hr after plating, were infected with recombinant adenovirus at moi of 50 in serum free media for three hrs. Forty-eight hrs after infection, the cells were lysed and luciferase levels were measured as above.

Luciferase measurement. Transiently transfected or adenovirus transfected cells were processed for luciferase activity using the Luc-Lite assay kit (Packard), as per manufacturer's instructions. Briefly, cells were washed in serum- and phenol red-free medium, then lysed in the presence of luciferase substrate to produce a long half-life “glow” luminescence. Relative light intensity was quantitated on a Fusion Universal Microplate Analyzer (Packard).

Transfection and fluorescent microscopy. Cells were transfected with HDAC5-GFP constructs and fixed with 3.7% formaldehyde at 48 hrs after transfection. GFP staining was observed by green fluorescence microscopy.

Isolation of stable transfectants. Cells were transfected with HDAC5-GFP plasmid containing the neo resistance marker in 100 mm culture plates with Fugene (Roche) per manufacturer's instructions. Three days after transfection the cells were placed under neo selection (800 μg/ml). Neo resistant colonies started to appear at about 2 weeks after transfection. GFP positive colonies were isolated by limited dilution.

NRVM Cytoblots and Immunocytochemistry. Ninety-six well gelatin-coated tissue culture plates were seeded with freshly isolated neonatal rat ventricular myocytes (NRVM) at a density of 10,000 cells per well. NRVM were maintained in Dulbecco's Modified Eagle's Medium (DMEM), supplemented with 10% fetal bovine serum (FBS). Twenty-four hours after plating, cells were placed in serum free media containing 20 uM phenylephrine (PE) to induce hypertrophy. Compounds were transferred onto wells as needed 30 min after PE addition using Biomek FX robotic liquid handling system. After compound addition, assay plates were allowed to incubate for a period of 48 hr, then prepared for cytoblot analysis. Assay plates were washed twice in phosphate buffered saline (PBS, 100 ul/well) then fixed in 4% paraformaldehyde in PBS for 30 min (100 ul/well).

Following two PBS washes, plates were blocked for 1 hr with a solution of 1% bovine serum albumin (BSA) in PBS (100 ul/well). Blocking solution was aspirated and replaced with appropriate primary antibody (rabbit polyclonal anti-S6 1:100, mouse monoclonal anti-ANP 1:200 or mouse monoclonal anti-actinin 1:200) in PBS+1% BSA, 50 ul/well for 1 hr. Assay plates were then aspirated, washed three times in PBS+1% BSA (100 ul/well) and secondary antibody was added (HRP-conjugated goat anti-rabbit for luminometry or Alexa 594 labeled goat anti mouse polyclonal for fluorescent microscopy, 1:500 dilution in PBS+1% BSA, 50 ul/well,) for 1 hr.

After incubation with secondary antibody, plates were aspirated, washed three times in PBS (100 ul/well), followed by addition of luminol reagent (Pierce SuperSignal, 50 ul/well) when HRP was used. Luminol treated plates were red on a Packard Fusion plate reader, which rapidly quantitates emitted light (<1 sec/well). Alexa 594 labeled plates were counterstained with Hoechst 33342 at 1 ug/ml in PBS and observed under fluorescent microscopy. Alexa 594 label was observed at 630 nm following excitation at 540 nm. Hoechst label was observed at 460 nm following excitation at 360 nm. Cell area was quantitated using the Metamorph (UIC) software package where the total area of Alexa 594 label was normalized to the number or nuclei in each field.

### Example 2

### Results

Comparison of 2-hyb assays in various cell lines. Mammalian two-hybrid assays test the ability of two proteins to interact in a mammalian cell of interest. One interacting protein is fused to the GAL4 DNA-binding domain, while the other is fused to the VP16 transactivation domain. The chimeric constructs are cotransfected with a GAL4 promoter-luciferase reporter construct; if the two proteins interact in vivo, the luciferase reporter is transcribed.

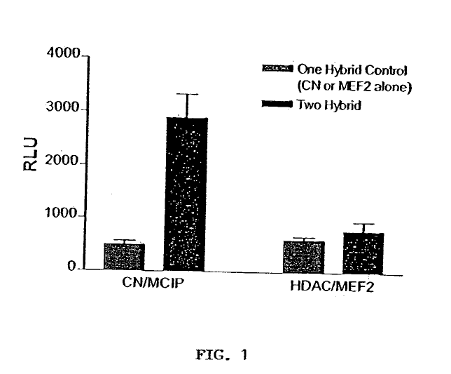

The inventors examined the interaction status of two pairs of proteins known to play a role in the regulation of cardiomyocyte hypertrophy: (1) the calcium-dependent regulatory phosphatase calcineurin (CN) and its endogenous inhibitor, myocyte-enriched calcineurin inhibitory protein (MCIP) and (2) the myocyte enhancer factor-2 transcription factor (MEF-2) and the MEF-2 inhibitor histone deacetylase (HDAC). In the course of this survey, the inventors observed that in H9C2 cells, while the CN/MCIP interaction was robust, the HDAC/MEF2 two-hybrid signal was extremely low (FIG. 1). This low reporter activity suggested that HDAC and MEF-2 interact weakly or are prevented from interacting in H9C2 cells. The low apparent interaction of HDAC and MEF-2 could be the result of a constitutive active HDAC kinase.

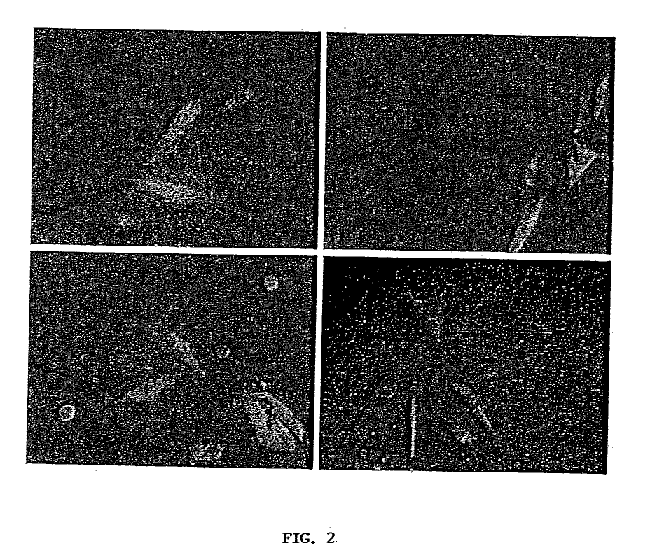

Cytoplasmic localization of H5-GFP. To determine the status of HDAC kinase, the inventors determined the subcellular localization of HDAC5-GFP using fluorescence microscopy. Following transient transfection of HDAC5-GFP, about 60% of viable cells showed a strictly cytoplasmic localization of GFP suggesting activated HDAC kinase. Similarly, in transfection experiments, the inventors were able to isolate four transfected colonies with all of these colonies showing cytoplasmic localization of GFP (FIG. 2).

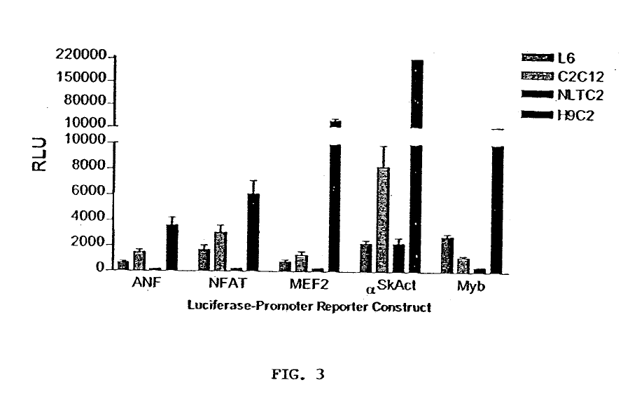

Activity of hypertrophic promoters in H9 as compared to other cell lines. To compare the relative degree of endogenous hypertrophic gene expression occurring in different cell lines, four myocyte lineages (L6, C2C12, NLTC2 and H9C2) were transfected with luciferase promoter-reporter constructs. Five promoters were examined: ANF, NFAT, MEF-2, alpha skeletal actin and myoglobin. Compared with other muscle lines, H9C2 cells were found to exhibit significantly higher endogenous levels of hypertrophic promoter activity than other lines examined (FIG. 3). The high endogenous activities of these reporters are consistent with the hypothesis that hypertrophic gene expression pathways are, to some degree, constitutively activated in H9C2 cells.

Effect of HDAC5, MCIP, FK506 and CsA by transfection. As mentioned above, there are two main pathways that could cause activation of the hypertrophic response, namely CN and CaMK. The CaMK pathway results in the de-repression of transcription factor MEF-2 due to the dissociation and nuclear export of HDAC4 and 5. To determine whether H9C2 cells can appropriately respond to known activators and repressors of hypertrophy, the inventors examined changes in reporter gene expression in response to a variety of gene products, calcineurin inhibitors and adrenergic agonists.

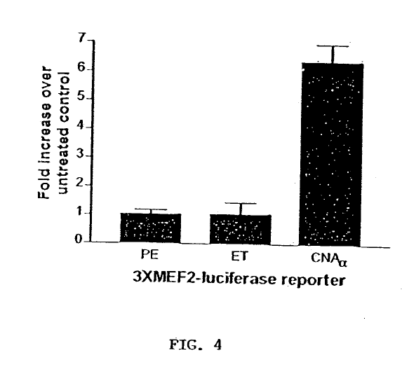

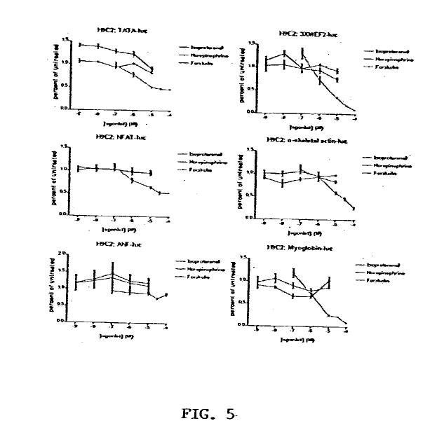

The inventors observed that expression of a constitutively active form of calcineurin A α, a known activator of cardiac hypertrophy, resulted in a six-fold increase in the basal expression of the MEF2 reporter (FIG. 4). The MEF-2 reporter was unaffected by treatment with the adrenergic agonists phenylephrine (PE, 100 μM) and endothelin (ET, 100 μM). A dose-response survey of other adrenergic agonists (isoproterenol, norepinephrine and forskolin) yielded similar results (FIG. 5); hypertrophic promoter activity in H9C2 cells appears to be relatively unaffected by agonist treatment.

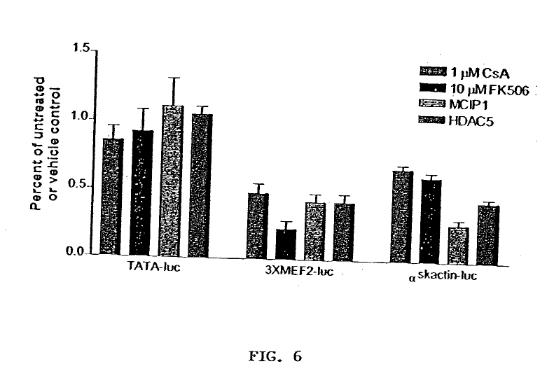

In contrast, treatment with the anti-hypertrophic calcineurin inhibitors cyclosporine (CsA) and FK506, or expression of the anti-hypertrophic gene products MCIP1 or HDAC, significantly reduced expression of MEF-2 and alpha skeletal actin reporters (FIG. 6).

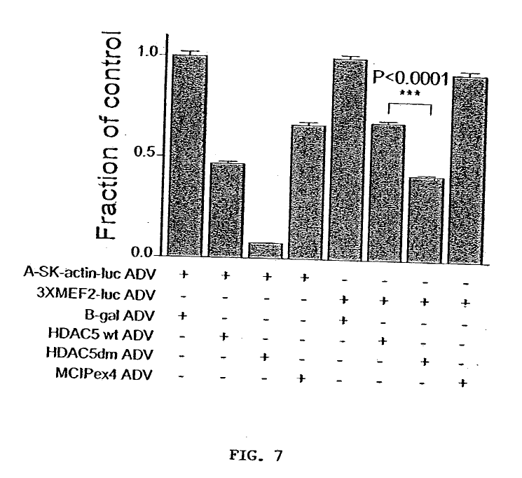

The effectiveness of MCIP1, HDAC and HDAC5-S259/498A (HDAC5dm) was evaluated again by recombinant adenovirus infection that results to virtually 100% gene transfer. HDAC5dm is a mutant of HDAC5 where the serine residues at 259 and 498 are replaced by alanine. This substitution removes the phosphorylation sites on HDAC5 that are necessary for the derepression of the MEF-2 pathway. Therefore, the HDAC5dm is a super-repressor of the MEF-2 pathway and it does not respond to activation signals. As seen in FIG. 7, all tested gene products repress both the 3×MEF2 promoter and the alpha-skeletal-actin promoter with HDAC5-S259/498A being the most effective repressor. These results are consistent with the hypothesis that the MEF-2 pathway could be responsible of the majority of the observed activity.

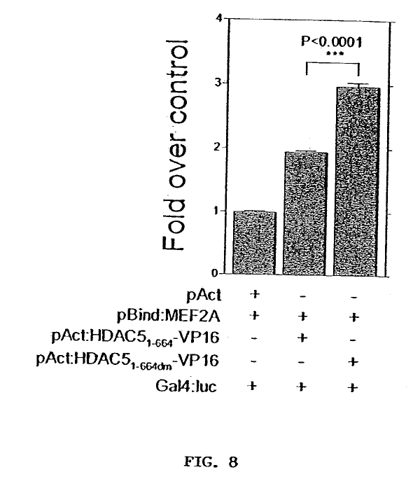

Effect of double mutation of HS-VP16 on 2-hyb signal If the MEF-2 pathway is activated in H9C2 cells, then the two hybrid signal from an HDAC5 construct carrying the S259/498A mutations is expected to be higher than the signal from the wild-type HDAC5. The results shown in FIG. 8 confirm this hypothesis. The HDAC5dm shows a slight but highly reproducible elevated signal over the HDAC5wt suggesting that the MEF-2 pathway is at least in part responsible for the observed endogenous activity of hypertrophic promoters.

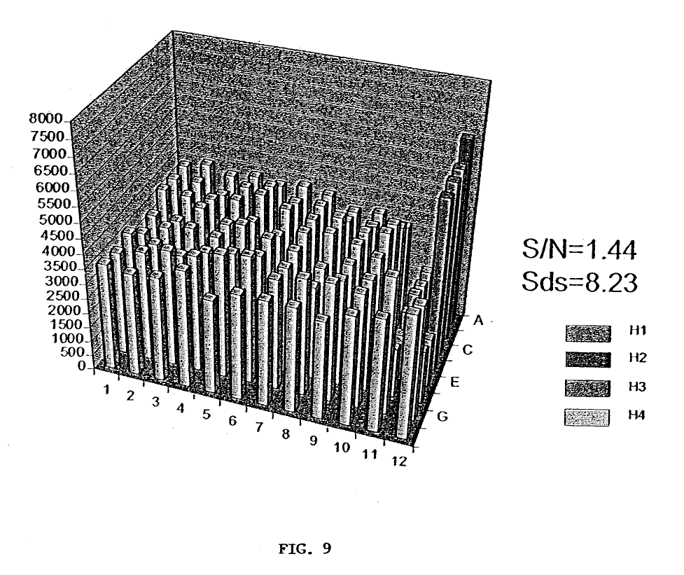

Example of assay based on HDAC/MEF-2. The inventors took advantage of the ability to modulate the MEF-2/HDAC interaction and built assays that can be used for screening of compounds that can render the MEF-2/HDAC interaction insensitive to hypertrophic signaling. These assays utilize the mammalian two-hybrid system to report the interaction between HDAC5 and MEF-2. In the original configuration of this assay in 10T1/2 or C2C12 cells, the inventors used constitutive active CaMKI to activate the MEF-2 pathway and lower the signal from the 2-hybrid system with the wt HDAC5. Compound screens are then set in the presence of CaMKI and compounds that elevate luciferase signal are scored as positive. The use of CaMKI complicates the assay and possibly restricts the searchable chemical space to CaMK inhibitors. The properties of H9 offer the opportunity to perform such a screening in a more “natural” environment and in the absence of exogenous activated CaMK. As discussed above, in H9C2 the two hybrid signal is lower than the expected signal if the hypertrophic pathways were not activated. Under non activation conditions the expected two hybrid signal is at least as high as the signal from the HDAC5dm. FIG. 9 shows a typical assay plate where wells A1-H10 are experimental samples with all components of the two hybrid system with wt HDAC. Wells A12-D12 are control samples using the two hybrid system with HDACdm. The reproducibility of the assay is such that positive hits are expected to be at least 8 standard deviations of the mean of negative samples.

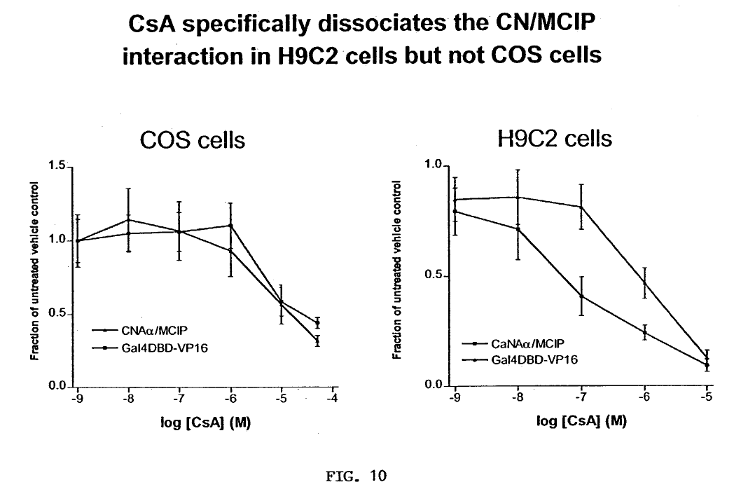

CsA Specifically Dissociates the CN/MCIP Interaction in H9C2 Cells but not COS Cells. Dose-response studies were initiated to determine the optimum dose of CsA which is able to specifically dissociate the mammalian two-hybrid CsA/MCIP1 interaction. Screening for compounds that restore the CN/MCIP interaction in the presence of CsA should yield muscle-specific CN inhibitors. COS or H9C2 cells were transiently cotransfected with a Gal4-luciferase reporter construct and either (i) Gal4DBD-CN and VP16-MCIP constructs or (ii) a non-dissociable Gal4DBD-VP16 fusion control construct. CsA was added at the time of transfection. Forty-eight hours after transfection, cells were processed for quantitation of luciferase activity. As shown in FIG. 10, in COS cells there is a non-specific loss of signal from the control (CsA toxicity) that occurs before CN/MCIP is dissociated, as the concentration of CsA increases. In H9C2 cells, however, treatment with 100 nM (10⁻⁷M) CsA permits ˜50% dissociation of CN/MCIP with little effect upon the control. These data indicate that H9C2 cells are uniquely suited for the development of certain assays, including the CN/MCIP two-hybrid assay.

Compound Screening Results. To evaluate further the usefulness of H9C2 cells in HT screening, the inventors applied an exploratory set of compounds as summarized in Table 1. The assay used was the MEF2/HDAC5 2-hybrid. First, the compounds were tested once at 10 μM on H9C2 cells transfected with the MEF2/HDAC5 2-hybrid components as described in the materials and methods. Hits from the primary assay were tested again in triplicate the same way as the secondary assay. Hits on the primary screen were defined as those compounds that increased luciferase levels at greater than 3 standard deviations above the mean of untreated controls. Hits on the secondary screen were compounds that increased luciferase levels at least sixty-six percent of the time at greater than 3 standard deviations above the mean of untreated controls. Of the 7680 compounds tested, 3.3% were scored positive on the primary assay and 34% of those scored positive on the secondary assay. The 87 confirmed secondary hits represent 1.1% of the starting library.

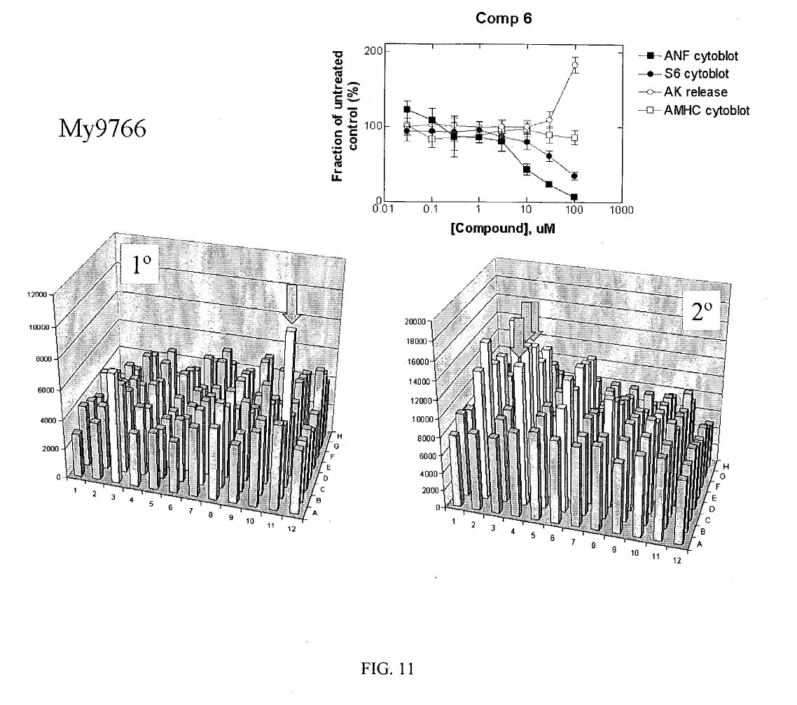

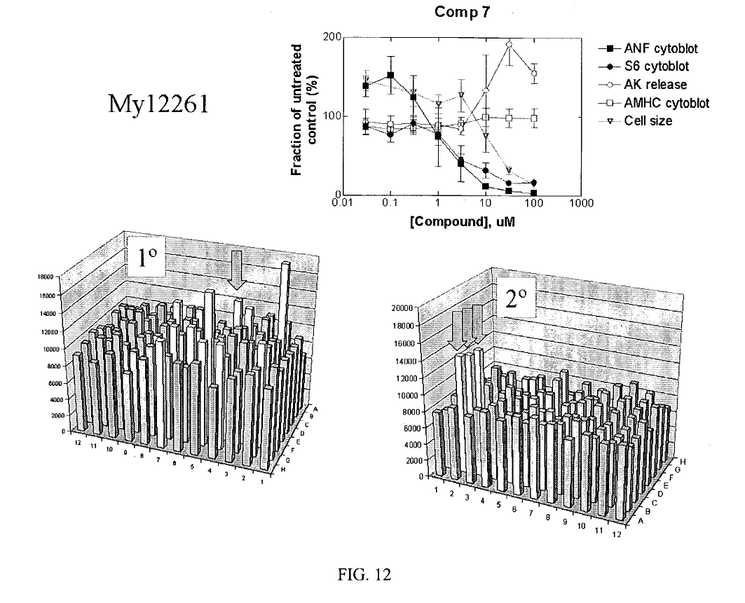

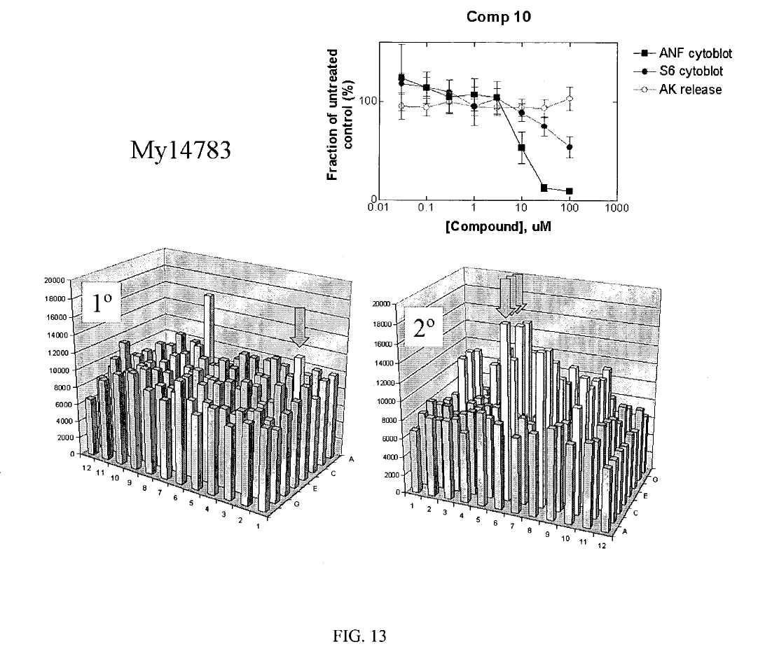

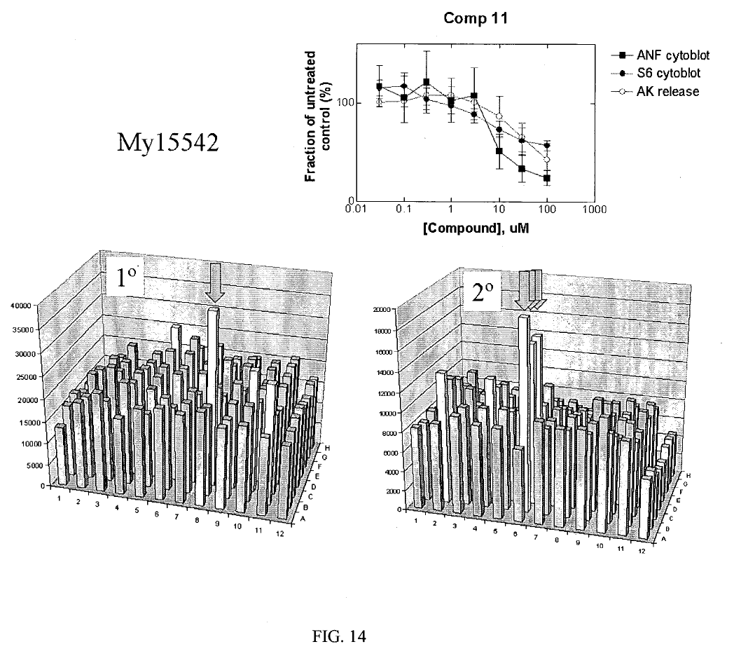

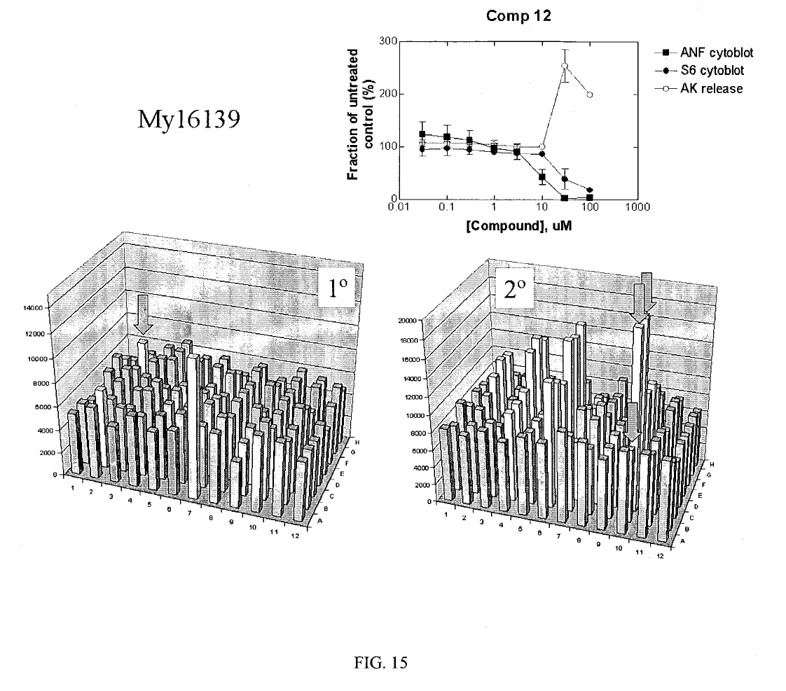

From the confirmed secondary hits, the inventors chose the 15 most potent and obtained dose response curves on the primary assay. About 2/3 of these showed well-behaved titration curves on the secondary assay. The same 15 compounds were also tested in hypertrophy assays on neonatal rat ventricular myocytes (NRVM). Initially, inventors determined the ability of these compounds to inhibit the ANF protein upregulation in the presence of PE measured using the cytoblot process as described in the materials and methods. Five compounds showed significant inhibition of the ANF protein upragulation on cytoblot assays (see FIGS. 11-15). These five compounds were studied further measuring additional hypertropic markers such as S6 protein or α-myocin heavy chain (αMHC) or cell area. In addition, cytotoxicity of these compounds was determined by measuring adenylated kinase (AK) release. Increased cytotoxicity is associated with elevation of AK in the cell media. With all compounds, the cytotoxicity curve was separated from at least one hypertophic marker titration curve. The results summarized in FIGS. 11 to 15 show results obtained with the 5 NRVM active compounds in the primary, secondary, and NRVM assays. These results clearly demonstrate that compounds that are selected on the basis of causing increase of the MEF2/HDAC 2-hybrid signal include compounds with anti-hypertrophic activity.

TABLE 1

| | Amount | % T | % G |
|---|---|---|---|
| Compounds screened | 7680 | | |
| 1° hits | 256 | 3.3 | |
| 2° hits | 87 | 1.1 | 34.0 |
| Compounds tested on NRVM | 15 | | |
| Active on NRVM | 5 | | 33.3 |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein, without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference.

U.S. Pat. No. 4,683,202

U.S. Pat. No. 4,797,368

U.S. Pat. No. 5,139,941

U.S. Pat. No. 5,288,173

U.S. Pat. No. 5,302,523

U.S. Pat. No. 5,322,783

U.S. Pat. No. 5,384,253

U.S. Pat. No. 5,464,765

U.S. Pat. No. 5,538,877

U.S. Pat. No. 5,538,880

U.S. Pat. No. 5,538,880

U.S. Pat. No. 5,550,318

U.S. Pat. No. 5,550,318

U.S. Pat. No. 5,563,055

U.S. Pat. No. 5,563,055

U.S. Pat. No. 5,580,859

U.S. Pat. No. 5,589,466

U.S. Pat. No. 5,591,616

U.S. Pat. No. 5,610,042

U.S. Pat. No. 5,610,042

U.S. Pat. No. 5,656,610

U.S. Pat. No. 5,702,932

U.S. Pat. No. 5,708,158

U.S. Pat. No. 5,736,524

U.S. Pat. No. 5,780,448

U.S. Pat. No. 5,789,215

U.S. Pat. No. 5,925,565

U.S. Pat. No. 5,928,906

U.S. Pat. No. 5,935,819

U.S. Pat. No. 5,945,100

U.S. Pat. No. 5,981,274

U.S. Pat. No. 5,994,136

U.S. Pat. No. 5,994,624

U.S. Pat. No. 6,013,516

Ausubel, Brent, Kingston, Moore, Seidman, Smith, Struhl, In: Current Protocols in Molecular Biology, John, Wiley & Sons, Inc, New York, 1994.

Blomer, Naldini, Kafri, Trono, Verma, Gage, “Highly efficient and sustained gene transfer in adult neurons with a lentivirus vector,” J Virol., 71(9):6641-6649, 1997.

Brand, “Myocyte enhancer factor 2 (MEF2),” Int J. Biochem. Cell Biol., 29:1467-1470; 1997.

Brostrom et al, Vasopressin-induced hypertrophy in H9c2 heart-derived myocytes. Int. J. Biochem. Cell. Biol. 32, 993-1006, 2000.

Carbonelli, Corley, Seigelchifer, Zorzopulos, “A plasmid vector for isolation of strong promoters in Escherichia coli,” FEMS Microbiol. Lett., 177(1):75-82, 1999.

Chandler, Mayeda, Yeakley, Kramer, Fu, “RNA splicing specificity determined by the coordinated action of RNA recognition motifs in SR proteins,” Proc, Nat'l Acad, Sci, USA, 94(8):3596-601, 1997.

Chen and Okayama, “High-efficiency transformation of mammalian cells by plasmid DNA,” Mol. Cell Biol., 7(8):2745-2752, 1987.

Chien, Bartel, Sternglanz, Fields, “The two-hybrid system: a method to identify and clone genes for proteins that interact with a protein of interest,” Proc. Nat'l Acad. Sci. USA, 88(21):9578-9582, 1991.

Cocea, “Duplication of a region in the multiple cloning site of a plasmid vector to enhance cloning-mediated addition of restriction sites to a DNA fragment,” Biotechniques, 23(5):814-816, 1997.

Cotten, Wagner, Zatloukal, Phillips, Curiel, Birnstiel, “High-efficiency receptor-mediated delivery of small and large (48 kilobase gene constructs using the endosome-disruption activity of defective or chemically inactivated adenovirus particles,” Proc. Nat'l Acad. Sci. USA, 89(13):6094-6098, 1992.

Coupar, Andrew, Boyle, “A general method for the construction of recombinant vaccinia viruses expressing multiple foreign genes,” Gene, 68:1-10, 1988.

Curiel, “High-efficiency gene transfer employing adenovirus-polylysine-DNA complexes,” Nat. Immun., 13(2-3):141-64, 1994.

EPO 0273085

Fechheimer, Boylan, Parker, Sisken, Patel, Zimmer, “Transfection of mammalian cells with plasmid DNA by scrape loading and sonication loading,” Proc. Nat'l Acad. Sci. USA, 84:8463-8467, 1987.

Fearon, Finkel, Gillison, Kennedy, Casella, Tomaselli, Morrow, VanDang, “Karyoplasmic interaction selection strategy: a general strategy to detect protein-protein interactions in manmalian cells,” Proc. Nat'l Acad. Sci. USA, 89(17):7958-62, 1992.

Fraley, Fomari, Kaplan, “Entrapment of a bacterial plasmid in phospholipid vesicles: potential for gene transfer,” Proc. Nat'l Acad. Sci. USA, 76:3348-3352, 1979.

Friedmann, “Progress toward human gene therapy,” Science, 244:1275-1281, 1989.

Ghosh and Bachhawat, Targeting of Liposomes to Hepatocytes. In: Liver Diseases, Targeted Diagnosis and Therapy Using Specific Receptors and Ligands. Wu et al., eds., Marcel Dekker, New York, pp. 87-104, 1991.

Gopal, “Gene transfer method for transient gene expression, stable transformation, and cotransformation of suspension cell cultures,” Mol. Cell Biol., 5:1188-1190, 1985.

Graham and van der Eb, “A new technique for the assay of infectivity of human adenovirus 5 DNA,” Virology, 52:456-467, 1973.

Grozinger, Hassig, Schreiber, “Three proteins define a class of human histone deacetylases related to yeast Hda1p,” Proc. Nat'l Acad. Sci. USA, 27;96(9):4868-73, 1999.

Grunhaus, Cho, Horwitz, “Association of vaccinia virus-expressed adenovirus E3-19K glycoprotein with class 1 MHC and its effects on virulence in a murine pneumonia model,” Seminar in Virology, 200(2):535-546, 1992.

Gyuris, Golemis, Chertkov, Brent., “Cdi1, a human GI and S phase protein phosphatase that associates with Cdk2,” Cell, 75(4):791-803, 1993.

Harland and Weintraub, “Translation of mRNA injected into Xenopus oocytes is specifically inhibited by antisense RNA,” J. Cell Biol., 101:1094-1099, 1985.

Haseloff, Siemering, Prasher, Hodge, “Removal of a cryptic intron and subcellular localization of green fluorescent protein are required to mark transgenic Arabidopsis plants brightly,” Proc. Nat'l Acad. Sci. USA, 94(6):2122-7, 1997.

Hescheler et al., Morphological, biochemical, and electrophysiological characterization of a clonal cell (H9c2) line from rat heart. Circ. Res. 69, 1476-86, 1991

Ho, Thomas, Timmerman, Li, Francke, Crabtree, “NFATc3, a lymphoid-specific NFATc family member that is calcium-regulated and exhibits distinct DNA binding specificity,” J. Biol. Chem., 270:19898-19907, 1995.

Hoey, Matthews, Badran, Pegg, Sillence, “Cardiovascular effects of clenbuterol are beta 2-adrenoceptor-mediated in steers,” J. Anim. Sci., 73(6):1754-1765, 1995.

Horwich, Furtak, Pugh, Summers, “Synthesis of hepadnavirus particles that contain replication-defective duck hepatitis B virus genomes in cultured HuH7 cells,” Virol., 64:642-650, 1990.

Kaneda, Iwai, Uchida, “Increased expression of DNA cointroduced with nuclear protein in adult rat liver,” Science, 243:375-378, 1989.

Kato et al., J. Biol. Chem., 266:3361-3364, 1991.

Kelleher and Vos, “Long-term episomal gene delivery in human lymphoid cells using human and avian adenoviral-assisted transfection,” Biotechniques, 17(6):1110-7, 1994.

Kimes and Brandt, “Properties of a clonal muscle cell line from rat heart,” Exp. Cell Res. 98, 367-81, 1976.

Klein et al., “High-velocity microprojectiles for delivering nucleic acids into living cells,” Nature, 327:70-73, 1987.

Laughlin, Cardellichio, Coon, “Latent infection of KB cells with adeno-associated virus type 2,” J. Virol., 60(2):515-24, 1986.

Lebkowski et al., Mol. Cell Biol., 8(10):3988-3996, 1988.

Levenson, Transue, Roninson, “Internal ribosomal entry site-containing retroviral vectors with green fluorescent protein and drug resistance markers,” Hum. Gene Ther., 9(8):1233-6, 1998.

McCaffery et al., Science, 262:750-754, 1993.

Maniatis, et al., Molecular Cloning, A Laboratory Manual, Cold Spring Harbor Press, Cold Spring Harbor, N.Y., 1989.

Mann, Mulligan, Baltimore, “Construction of a retrovirus packaging mutant and its use to produce helper-free defective retrovirus,” Cell, 33:153-159, 1983.

Masuda, Naito, Tokumitsu, Campbell, Saito, Hannum, Arai, Arai “NFATx, a novel member of the nuclear factor of activated T cells family that is expressed predominantly in the thymus,” Mol. Cell Biol., 15(5):2697-706, 1995.

McLaughlin, Collis, Hermonat, Muzyczka, “Adeno-associated virus general transduction vectors: analysis of proviral structures,” J. Virol., 62(6):1963-73, 1988.

Miller, Hill, Slovak, Shimm, “Radiation resistance in a doxorubicin-resistant human fibrosarcoma cell line,” Am. J. Clin. Oncol., 15(3):216-21, 1992.

Muzyczka, “Use of adeno-associated virus as a general transduction vector for mammalian cells,” Curr. Top. Microbiol. Immunol., 158:97-129, 1992.

Naldini, Blomer, Gallay, Ory, Mulligan, Gage, Verma, Trono, “In vivo gene delivery and stable transduction of nondividing cells by a lentiviral vector,” Science, 272(5259):263-267, 1996.

Nicolas and Rubinstein, In: Vectors: A survey of molecular cloning vectors and their uses, Rodriguez and Denhardt, eds., Stoneham: Butterworth, pp. 494-513, 1988.

Nicolau and Sene, “Liposome-mediated DNA transfer in eukaryotic cells. Dependence of the transfer efficiency upon the type of liposomes used and the host cell cycle stage,” Biochim. Biophys. Acta, 721:185-190, 1982.

Nicolau and Sene, “Liposome-mediated DNA transfer in eukaryotic cells. Dependence of the transfer efficiency upon the type of liposomes used and the host cell cycle stage,” Biochim. Biophys. Acta, 721:185-190, 1982.

Nicolau, Legrand, Grosse, “Liposomes as carriers for in vivo gene transfer and expression,” Methods Enzymol., 149:157-176, 1987.

Northrop, Ho, Chen, Thomas, Timmerman, Nolan, Admon, Crabtree, “NF-AT components define a family of transcription factors targeted in T-cell activation,” Nature, 369(6480):497-502, 1994.

Olson, Perry, Schulz “Regulation of muscle differentiation by the MEF2 family of MADS box transcription factors,” Dev. Biol., 172(1):2-14, 1995.

Omirulleh, Abraham, Golovkin, Stefanov, Karabaev, Mustardy, Morocz, Dudits, “Activity of a chimeric promoter with the doubled CaMV 35S enhancer element in protoplast-derived cells and transgenic plants in maize,” Plant Mol. Biol., 21(3):415-28, 1993.

Park, Takeuchi, Sharma, “Characterization of a new isoform of the NFAT (nuclear factor of activated T cells) gene family member NFATc,” J. Biol. Chem., 271(34):20914-20921, 1996.

Paskind, Weinberg, Baltimore, “Dependence of Moloney murine leukemia virus production on cell growth,” Virology, 67:242-248, 1975.

PCT Application No. WO 94/09699

PCT Application No. WO 94/09699

PCT Application No. WO 95/06128

PCT Application No. WO 97/41228

Pelletier and Sonenberg, “Internal initiation of translation of eukaryotic mRNA directed by a sequence derived from poliovirus RNA,” Nature, 334(6180):320-325, 1988.

Perales, Ferkol, Beegen, Ratnoff, Hanson, “Gene transfer in vivo: sustained expression and regulation of genes introduced into the liver by receptor-targeted uptake,” Proc. Nat'l Acad. Sci. 91:4086-4090, 1994.

Potrykus, Paszkowski, Saul, Petruska, Shillito, “Molecular and general genetics of a hybrid foreign gene introduced into tobacco by direct gene transfer,” Mol. Gen. Genet., 199(2):169-77, 1985.

Potter, Weir, Leder, “Enhancer-dependent expression of human kappa immunoglobulin genes introduced into mouse pre-B lymphocytes by electroporation,” Proc. Nat'l Acad. Sci. USA, 81:7161-7165, 1984.

Reichel, Mathur, Eckes, Langenkemper, Koncz, Schell, Reiss, Maas, “Enhanced green fluorescence by the expression of an Aequorea victoria green fluorescent protein mutant in mono- and dicotyledonous plant cells,” Proc. Nat'l Acad. Sci. USA, 93(12):5888-93, 1996.

Ridgeway, “Mammalian expression vectors,” In: Vectors: A survey of molecular cloning vectors and their uses. Rodriguez and Denhardt, eds. Stoneham: Butterworth, pp. 467-492, 1988.

Rippe, Brenner and Leffert, “DNA-mediated gene transfer into adult rat hepatocytes in primary culture,” Mol. Cell Biol., 10:689-695, 1990.

Rooney, Hodge, McCaffrey, Rao, Glimcher, “A common factor regulates both Th1- and Th2-specific cytokine gene expression,” EMBO J., 13(3):625-33, 1994.

Roux, Jeanteur, Piechaczyk, “A versatile and potentially general approach to the targeting of specific cell types by retroviruses: Application to the infection of human cells by means of major histocompatibility complex class I and class II antigens by mouse ecotropic munne leukemia virus-derived viruses,” Proc. Nat'l Acad. Sci. USA, 86:9079-9083, 1989.

Sambrook et. al., In: Molecular Cloning: A Laboratory Manual, 2d Ed., Cold Spring Harbor Laboratory Press, Cold Spring Harbor, N.Y., 1989.

Sheen, Hwang, Niwa, Kobayashi, Galbraith, “Green-fluorescent protein as a new vital marker in plant cells,” Plant J., 8(5):777-84, 1995.

Temin, “Retrovirus vectors for gene transfer: Efficient integration into and expression of exogenous DNA in vertebrate cell genome,” In: Gene Transfer, Kucherlapati (ed.), New York: Plenum Press, pp. 149-188, 1986.

Tian, McKnight, Russell, “Endothelial PAS domain protein 1 (EPAS 1), a transcription factor selectively expressed in endothelial cells,” Genes Dev., 11(1):72-82, 1997.

Tratschin, West, Sandbank, and Carter, “A human parvovirus, adeno-associated virus, as a eucaryotic vector: transient expression and encapsidation of the procaryotic gene for chloramphenicol acetyltransferase,” Mol. Cell. Biol., 4:2072-2081, 1984.

Tur-Kaspa, Teicher, Levine, Skoultchi and Shafritz, “Use of electroporation to introduce biologically active foreign genes into primary rat hepatocytes,” Mol. Cell Biol., 6:716-718, 1986.

Vasavada, Ganguly, Germino, Wang, Weissman, “A contingent replication assay for the detection of protein-protein interactions in animal cells,” Proc. Nat'l Acad. Sci. USA, 88(23):10686-90, 1991.

Wagner, Zenke, Cotten, Beug, Bimstiel, “Transferrin-polycation conjugates as carriers for DNA uptake into cells,” Proc. Nat'l Acad. Sci. USA 87(9):3410-3414, 1990.

Workman and Kingston, “Alteration of nucleosome structure as a mechanism of transcriptional regulation,” Annu. Rev. Biochem., 67:545-79, 1998. Wong, Nicolau, Hofschneider, “Appearance of β-lactamase activity in animal cells upon liposome mediated gene transfer,” Gene, 10:87-94, 1980.

Wu and Wu, “Receptor-mediated in vitro gene transfections by a soluble DNA carrier system,” J. Biol. Chem., 262:4429-4432, 1987.

Wu and Wu, “Evidence for targeted gene delivery to HepG2 hepatoma cells in vitro,” Biochemistry, 27:887-892, 1988.

Wu and Wu, Adv. Drug Delivery Rev., 12:159-167, 1993.

Zufferey, Nagy, Mandel, Naldini, Trono, “Multiply attenuated lentiviral vector achieves efficient gene delivery in vivo,” Nat. Biotechnol., 15(9):871-875, 1997.

What is claimed is:

1. A method of identifying an anti-hypertrophic agent comprising:
(a) providing an H9C2 cell comprising an expression cassette, said expression cassette comprising a hypertrophic promoter operably linked to a reporter coding region;
(b) contacting said cell with a candidate substance; and
(c) measuring the effect of said candidate substance on the activity of the hypertrophic promoter,
wherein a reduction in the activity of the hypertrophic promoter, as compared to hypertrophic promoter activity in an H9C2 cell not treated with said candidate substance, indicates that said candidate substance is an anti-hypertrophic agent.

2. The method of claim 1, wherein said hypertrophic promoter is selected from the group consisting of ANF, α-skeletal actin, myoglobin, α-myosin heavy chain, β-myosin heavy chain, NFAT and MEF-2.

3. The method of claim 1, wherein said reporter coding region comprises a coding region for luciferase, green fluorescent protein, β-galactosidase, chloramphenicol acetyl transferase and secreted alkaline phosphatase.

4. The method of claim 1, wherein said cell is stably transformed with said expression cassette.

5. The method of claim 1, wherein said cell is transiently transformed with said expression cassette.

6. The method of claim 1, wherein said candidate substance is a polynucleotide or an oligonucleotide.

7. The method of claim 6, wherein said nucleic acid is a DNA molecule or an RNA molecule.

8. The method of claim 1, wherein said candidate substance is a polypeptide or a peptide.

9. The method of claim 1, wherein said candidate substance is a small molecule.

10. The method of claim 1, further comprising measuring the activity of said hypertrophic promoter in the absence of said candidate substance.

11. A method of identifying an anti-hypertrophic agent comprising:
(a) providing an H9C2 cell comprising an expression cassette, said expression cassette comprising a promoter active in said cell operably linked to a coding region for an HDAC4 or 5-GFP fusion polypeptide or an NFAT-GFP fusion polypeptide;
(b) contacting said cell with a candidate substance; and
(c) measuring the effect of said candidate substance on the nuclear localization of GFP fluorescence,
wherein an increase in nuclear localization of GFP fluorescence, as compared to nuclear localization of GFP fluorescence in an H9C2 cell not treated with said candidate substance, indicates that said candidate substance is an anti-hypertrophic agent.

12. The method of claim 11, wherein said promoter is CMV IE or SV40 large T.

13. The method of claim 11, wherein said coding region encodes an HDAC4, 5, 7 or 9-GFP fusion polypeptide.

14. The method of claim 11, wherein said coding region encodes an NFAT-GFP fusion polypeptide.

15. The method of claim 11, wherein said cell is stably transformed with said expression cassette.

16. The method of claim 11, wherein said cell is transiently transformed with said expression cassette.

17. The method of claim 11, wherein said candidate substance is a polynucleotide or an oligonucleotide.

18. The method of claim 17, wherein said nucleic acid is a DNA molecule or an RNA molecule.

19. The method of claim 11, wherein said candidate substance is a polypeptide or a peptide.

20. The method of claim 11, wherein said candidate substance is a small molecule.

21. The method of claim 11, further comprising measuring the nuclear localization of GFP fluorescence in the absence of said candidate substance.

22. A method of identifying an anti-hypertrophic agent comprising:
(a) providing an H9C2 cell comprising
(i) a first expression cassette, said first expression cassette comprising a GAL4 promoter operably linked to a reporter coding region;
(ii) a second expression cassette, said second expression cassette comprising a promoter active in said cell operably linked to a coding region for a MEF-2 fusion protein, wherein the MEF-2 fusion partner is VP16 or GAL4; and
(iii) a third expression cassette, said third expression cassette comprising a promoter active in said cell operably linked to a coding region for an HDAC4 or 5 fusion protein, wherein the HDAC4 or 5 fusion partner is VP16 or GAL4, but different from the fusion partner for MEF-2,
(b) contacting said cell with a candidate substance; and
(c) measuring the effect of said candidate substance on the activity of said GAL4 promoter,
wherein an increase in the activity of said GAL4 promoter, as compared to GAL4 promoter activity in an H9C2 cell not treated with said candidate substance, indicates that said candidate substance is an anti-hypertrophic agent.

23. The method of claim 22, wherein said promoter is CMV IE or SV40 large T.

24. The method of claim 22, wherein HDAC4, 5, 7 or 9 is fused to GAL4 and MEF-2 is fused to VP16.

25. The method of claim 22, wherein MEF-2 is fused to GAL4 and HDAC4, 5, 7 or 9 is fused to VP16.

26. The method of claim 22, wherein said cell is stably transformed with at least one of said expression cassettes.

27. The method of claim 26, wherein said cell is stably transformed with all of said expression cassettes.

28. The method of claim 22, wherein said cell is transiently transformed with at least one of said expression cassettes.

29. The method of claim 28, wherein said cell is transiently transformed with all of said expression cassettes.

30. The method of claim 22, wherein said candidate substance is a polynucleotide or an oligonucleotide.

31. The method of claim 30, wherein said nucleic acid is a DNA molecule or an RNA molecule.

32. The method of claim 22, wherein said candidate substance is a polypeptide or a peptide.

33. The method of claim 22, wherein said candidate substance is a small molecule.

34. The method of claim 22, further comprising measuring the GAL4 activity in the absence of said candidate substance.
